SEQUENCE LISTING (11 sequences)

SEQ ID NO: 1 - DNA, 17 bp, Artificial
  CGT_F
ctcgagatggcgctgct

SEQ ID NO: 2 - DNA, 30 bp, Artificial
  CGT_R
tctagattatacatctaggatttcctctgc

SEQ ID NO: 3 - DNA, 1185 bp, Artificial
  Cricetulus griseus ceramide glucosyltransferase_mRNA
atggcgctgctggacctggcccaggagggaatggccttgttcggcttcgtgctcttcgtggtgctgtggctgatgcatttcatgtccatcatctacacccggttacacctcaacaagaaggcaacagacaagcagccgtatagcaagctccctggtgtctctcttctgaaaccattgaagggggtggatcctaacctaatcaacaacttggagacattctttgaactggattatcccaaatatgaagtactcctttgtgtacaagatcatgatgatccagccattgatgtatgtaagaaactgcttggaaaatacccgaatgttgatgctagactatttataggtggcaaaaaggttggcattaatcctaaaattaataatttgatgccaggatatgaagttgcaaaatatgatctcatatggatttgtgatagtggaataagagtcattccagatacattaactgacatggtcaatcaaatgacggagaaggtgggcttggttcatgggctgccttatgtagcggacagacagggctttgctgccacattggaacaggtatattttggaacttcacacccaagatcctatatctctgccaatgtcactggcttcaaatgtgtgacgggaatgtcttgtttaatgaggaaggatgtgctagatcaggccggagggctgatagcctttgctcagtacattgctgaagattactttatggccaaagcaatagctgaccgaggttggcggtttgcaatgtctactcaagttgccatgcaaaactctggttcatactcaatttctcagtttcaatccagaatgatcaggtggaccaaattgagaattaacatgcttcctgctacaataatttgtgagccaatttcagaatgctttgttgccagtttaattattgggtgggcagcccaccatgtgttcagatgggatatcatggtcttcttcatgtgccattgcctagcatggtttatatttgactacattcaactaaggggtgtccagggtggcacactgtgtttttcaaaacttgattatgctgtggcttggttcatccgagaatccatgacaatatacattttcttgtcggcattatgggaccctactataagctggagaactggtcgctacaggttgcgttgtggaggaacagcagaggaaatcctagatgtataa

SEQ ID NO: 4 - RNA, 21 nt, Artificial
  Sense-siRNA_675
ugauagccuuugcucaguaca

SEQ ID NO: 5 - RNA, 21 nt, Artificial
  Antisense-siRNA_675
uguacugagcaaaggcuauca

SEQ ID NO: 6 - RNA, 21 nt, Artificial
  Sense-siRNA_926
guguucagaugggauaucaug

SEQ ID NO: 7 - RNA, 21 nt, Artificial
  Antisense-siRNA_926
caugauaucccaucugaacac

SEQ ID NO: 8 - RNA, 21 nt, Artificial
  Sense-siRNA_1098
cauuaugggacccuacuauaa

SEQ ID NO: 9 - RNA, 21 nt, Artificial
  Antisense-siRNA_1098
uuauaguagggucccauaaug

SEQ ID NO: 10 - DNA, 64 bp, Artificial
  CGT-1098_F
tgctgttatagtagggtcccataatggttttggccactgactgaccattatggccctactataa

SEQ ID NO: 11 - DNA, 64 bp, Artificial
  CGT-1098_R
cctgttatagtagggccataatggtcagtcagtggccaaaaccattatgggaccctactataac

(19) United States
(12) Patent Application Publication
(10) Pub. No.: US 2017/0129932 A1
(43) Pub. Date: May 11, 2017

(54) METHOD FOR PREPARING RECOMBINANT GLYCOPROTEIN HAVING HIGH SIALIC ACID CONTENT, THROUGH GLYCOSPHINGOLIPID SYNTHESIS CYCLE CONTROL

(71) Applicant: KOREA ADVANCED INSTITUTE OF SCIENCE AND TECHNOLOGY, Daejeon (KR)

(72) Inventors: Jung-Hoe KIM, Daejeon (KR); Chan-Yeong KWAK, Daejeon (KR); Seung-Yeol PARK, Gyeonggi-do (KR); Woo-Yong SHIM, Daejeon (KR)

(21) Appl. No.: 15/319,308

(22) PCT Filed: Sep. 24, 2014
(86) PCT No.: PCT/KR2014/008896

§ 371 Date: Dec. 15, 2016

(30) Foreign Application Priority Data
Jun. 18, 2014 (KR) 10-2014-0074095
Jul. 18, 2014 (KR) 10-2014-0090992

(51) Int. Cl. C07K 14/505; C12N 15/113; C12P 21/00

(52) U.S. Cl. CPC C07K 14/505; C12P 21/005; C12N 15/1137; C12Y 204/0108; C12N 2310/14; C12N 2310/141

(57) ABSTRACT

The present invention relates to a method for preparing a recombinant cell line producing recombinant glycoproteins having a high sialic acid content by inhibiting glycosphingolipid (GSL) biosynthesis pathway in a cell line producing recombinant glycoproteins. Particularly, the present invention produces erythropoietin (EPO), containing a high content of sialic acid in a cell, from a cell line, which induces CGT inhibition by using siRNA and miRNA specifically binding to ceramide glucosyltransferase (CGT), thereby increasing the in vivo half-life of a recombinant therapeutic protein, and thus can be useful in the treatment of disease using the same.

### BACKGROUND OF THE INVENTION

1. Field of the Invention

The present invention relates to a method for preparing glycoproteins with increased sialic acid content by regulating glycosphingolipid biosynthesis pathway by inhibiting ceramide glucosyltransferase (CGT).

2. Description of the Related Art

Glycosylation is an important post-translational modification in mammalian cells, which is divided into N-linked and O-linked glycosylations. N-linked glycosylation happens on Asn (Asn-X-Ser/Thr motif) in many glycoproteins. Glycosylation affects enzyme activity, protein stability, and immunogenicity. N-acetylneuraminic acid and sialic acid are charged sugars and are attached to terminal galactose by α2-3/6 glycosidic linkage. This seems to affect in vivo half-life of glycoproteins discharged in order to prevent the recognition of the second galactose from asialoglycoprotein receptor (ASGPR) for the decomposition in liver cells. Since sialic acid regulates the stability of in vivo glycoproteins, raising the content of sialic acid in recombinant therapeutic proteins has been an important issue for the past 10 years.

Sialic acid is an essential component in the structure of a sugar precursor. Sugar chain precursors attached with approximately 50 kinds of sialic acids have been identified in the natural world so far. Sialic acid is acting as a precursor who can determine the signaling in cells and controlling the intercellular interaction in mammals, and plays an important role in the intracellular biological phenomena including the stability of glycoproteins etc. The in vivo sialic acid has been known as one of the first recognized sugar chains after infection of a pathogen (Sasisekharan and Myette, Am. Sci. 91:432-441(2003); Vimr and Lichtensteiger, Trends Microbiol. 10:254-257(2002)). The sialic acid on the cell surface is known as a component which forms polysugar oligosaccharides in the form of a capsule for immune evasion or to protect cells in a pathogenic microorganism itself (Vimr et al., Microbiol. Mol. Biol. Rev. 68:132-153(2004)). In those pathogenic microorganisms that can synthesize sialic acid in themselves, among all the pathogenic microorganisms, sialic acid is synthesized through sialic acid metabolic pathway. Or, extracellular sialic acid is transferred into a cell by the sialic acid transporter existing on the cell surface, leading to sialic acid metabolic pathway to produce sialic acid (Vimr and Lichtensteiger, Trends Microbiol. 10:254-257(2002)). In other cases, some microorganisms cut out sialic acid of a host or from the extracellular sialic acid sugar chain precursor by using trans-sialidase and then add it to the sugar chain on the cell surface (Scudder et al., Patent No. 10-1083065 J. Biol. Chem. 268:9886-9891(1993)). Trans-sialidase has the trans-glycosidase activity and is the enzyme playing a role in transporting sialic acid to various monosaccharides including galactose, disaccharides, and oligosaccharides from sialyllactose.

CMP-Neu5Ac acts as a substrate in the course of sialic acid biosynthesis. Sialylation is delivered into the luminal side of Golgi by CMP-Neu5Ac transporter (CSAT) in a glycosylation carrier, and then the delivered sialic acid is attached to terminal galactose by sialyl transferase (ST). The increase of sialic acid content in a recombinant therapeutic protein is accelerated by the increase of a potent N-linked glycosylation region. Also, the increase of sialylation of a recombinant therapeutic protein is promoted by the over-expression of a core enzyme for sialic acid biosynthesis such as galactosyltransferase (GT) or sialyl transferase (ST). CMP-Neu5Ac functioning as a restrictive factor for sialylation is increased in the luminal side of Golgi when ManNAc is supplemented or CST is over-expressed.

Glycosphingolipid (GSL) is another form of a glycosylation carrier expressed on cell surface, and plays an important role in cell growth, cell adhesion, and signal transduction. Glycosphingolipid is divided according to the glycan structure as follows; ganglioseries (GlcNAcβ4Galβ4GlcβCer), globoseries (Galα4Galβ4GlcβCer), lactoseries (GlcNAcβ3Galβ4GlcβCer), and neo-lactoseries (Galβ4GlcNAcβ3Galβ4GlcβCer). GSL synthesis begins when ceramide is delivered into cis-Golgi by CERT through a non-follicular transporter. GlcCer (glucosyl cerimide), the GSL precursor, is produced by ceramide glucosyltransferase (CGT). The inhibition of ceramide glucosyltransferase by using EtDO-P4 (chemical inhibitor) or RNAi suppressing glycosphingolipid synthesis causes cell growth and migration. Neutral glycosphingolipid is transported to trans-Golgi by FAPP2, while sialylated glycosphingolipid is synthesized in Golgi cisternae. CMP-Neu5Ac is used not only for the sialylation of glycoproteins but also for the synthesis of glycosphingolipids such as ganglioside.

The present inventors tried to develop a recombinant glycoprotein containing a high content of sialic acid. As a result, the present inventors succeeded in preparing a cell line inducing CGT inhibition by using a ceramide glucosyltransferase (CGT) inhibitor involved in glycosphingolipid biosynthesis pathway or siRNA and miRNA specifically binding to the same and confirmed the production of erythropoietin (EPO) containing a high content of sialic acid in the cell line, leading to the completion of the present invention.

### SUMMARY OF THE INVENTION

It is an object of the present invention to provide a method for preparing a recombinant cell line producing recombinant glycoproteins having a high sialic acid content which comprises the step of inhibiting glycosphingolipid (GSL) biosynthesis pathway in the cell line producing glycoproteins.

It is another object of the present invention to provide a recombinant cell line producing recombinant glycoproteins having a high sialic acid content wherein the glycosphingolipid biosynthesis pathway is suppressed.

In addition, it is also an object of the present invention to provide a method for preparing a recombinant glycoprotein having a high sialic acid content which comprises the following steps:

1) culturing the recombinant cell line of the present invention; and,

2) separating a glycoprotein having high sialic acid content from the culture solution of step 1).

It is further an object of the present invention to provide a use of the recombinant cell line producing recombinant glycoproteins having a high sialic acid content wherein the glycosphingolipid biosynthesis pathway is suppressed.

To achieve the above objects, the present invention provides a method for preparing a recombinant cell line producing a recombinant glycoprotein having a high sialic acid content which comprises the step of inhibiting glycosphingolipid (GSL) biosynthesis pathway in the cell line producing recombinant glycoproteins.

The present invention provides a recombinant cell line producing a recombinant glycoprotein having a high sialic acid content wherein the glycosphingolipid biosynthesis pathway is suppressed.

In addition, the present invention provides a method for preparing a recombinant glycoprotein having a high sialic acid content which comprises the following steps:

1) culturing the recombinant cell line of the present invention; and,

2) separating a glycoprotein having high sialic acid content from the culture solution of step 1).

The present invention also provides a use of the recombinant cell line producing recombinant glycoproteins having a high sialic acid content wherein the glycosphingolipid biosynthesis pathway is suppressed.

### Advantageous Effect

The present invention provides a cell line inducing CGT inhibition by using siRNA and miRNA specifically binding to ceramide glucosyltransferase (CGT), such as EC2-1H9-miR1 or EC2-1H9-miR2. The cell line can produce EPO containing a high content of sialic acid in the cells, so that it can increase the stability of recombinant therapeutic proteins, suggesting that it can be effectively used for the prevention and treatment of disease.

### BRIEF DESCRIPTION OF THE DRAWINGS

The application of the preferred embodiments of the present invention is best understood with reference to the accompanying drawings, wherein:

FIG. 1 inclusive of views A, B and C is a diagram illustrating the changes in the contents of GSL and sialic acid in EC2-1H9, the cell line producing recombinant human EPO, according to the treatment of EtDO-P4.

FIG. 2 inclusive of views A, B and C is a diagram illustrating the changes in the contents of GSL and sialic acid in EC2-1H9 according to the treatment of siRNA inhibiting ceramide glucosyltransferase (CGT).

FIG. 3 inclusive of views A, B and C is a diagram illustrating the changes in the contents of GSL and sialic acid in the stable cells transfected with pcCGT-miRNA.

FIG. 4 inclusive of views A and B is a diagram illustrating the CMP-NeuNAc content in the stable cells transfected with pcCGT-miRNA and the sialic acid content in EPO.

FIG. 5 inclusive of views A and B is a diagram illustrating cell growth and EPO production in the stable cells transfected with pcCGT-miRNA.

### DESCRIPTION OF THE PREFERRED EMBODIMENTS

Hereinafter, the present invention is described in detail.

The present invention provides a method for preparing a recombinant cell line producing a recombinant glycoprotein having a high sialic acid content which comprises the step of inhibiting glycosphingolipid (GSL) biosynthesis pathway in the cell line producing recombinant glycoproteins.

The glycoprotein herein is preferably selected from the group consisting of erythropoietin, thrombopoietin, alpha-antitrypsin, cholinesterase, chorionic gonadotropin, CTLA4Ig, Factor □, gammaglutamyltransferase, granulocyte colony-stimulating Factor (G-CSF), and luteinizing hormone (LH) which are suitable for the conjugation with sialic acid, and more preferably either erythropoietin or thrombopoietin, but not always limited thereto.

The inhibition of glycosphingolipid biosynthesis pathway is preferably achieved by inhibiting ceramide glucosyltransferase (CGT) which is composed of the amino acid sequence represented by SEQ. ID. NO: 3.

The inhibition of ceramide glucosyltransferase is preferably achieved through the treatment of a ceramide glucosyltransferase inhibitor or through the transfection with any sequence selected from the group consisting of antisense nucleotide, siRNA, shRNA, and miRNA binding to ceramide glucosyltransferase mRNA.

The ceramide glucosyltransferase inhibitor herein is preferably selected from the group consisting of P4 (D-threo-1-phenyl-2-palmitoylamino-3-pyrrolidino-1-propanol), EtDO-P4 (D-threo-1-(3′,4′-ethylenedioxy)phenyl-2-palmitoylamino-3-pyrrolidino-1-propanol), and pOH-P4 (D-threo-4′-hydroxy-1-phenyl-2-palmitoylamino-3-pyrrolidino-1-propanol), but not always limited thereto.

The said siRNA is preferably composed of one of those nucleotide sequences represented by SEQ. ID. NO: 4˜NO: 9, but not always limited thereto. The said miRNA is preferably composed of one of those nucleotide sequences represented by SEQ. ID. NO: 10˜NO: 11, but not always limited thereto.

The said siRNA can contain an independent sense RNA strand having homology with the target sequence above and an antisense RNA strand corresponding thereto, or can be a single RNA strand having a stem-loop structure wherein the sense RNA strand and the antisense RNA strand are connected by the loop.

The siRNA above not just has the structure made of a double-strand RNA composed of a complete pair of RNAs but also has a hairpin structure composed of a stem-loop structure which is particularly called shRNA (short hairpin RNA). The double-strand or stem can contain a region that is not able to be paired by mismatch (the corresponding nucleotide is not complementary) or bulge (having no nucleotide corresponding to one strand). The total length of the said siRNA is preferably 10˜80 bp long, more preferably 15˜60 bp long, and most preferably 20˜bp long. The loop sequence does not matter but preferably is 3˜10 bp long in order to keep a proper distance between the sense sequence and the antisense sequence in the link. Example sequences of the siRNA loop are as follows: AUG (Sui et al., Proc. Natl. Acad. Sci. USA 99(8):5515-5520, 2002), CCC, CCACC or CCACACC (Paul et al., Nature Biotechnology 20:505-508, 2002), UUCG (Lee et al., Nature Biotechnology 20:500-505), CTCGAG, AAGCUU (Editors of Nature Cell Biology Whither RNAi, Nat Cell Biol. 5:489-490, 2003), UUCAAGAGA (Yu et al., Proc. Natl. Acad. Sci. USA 99(9):6047-6052, 2002), and TTGATATCCG (www.genscript.com [symbol] default spacer). The siRNA can have a blunt end or a cohesive end. The cohesive end is either 3′ end protruding structure or 5′ end protruding structure, and the number of protruding nucleotides is not limited. For example, the number of protruding nucleotides is 1˜8 or preferably 2˜6. The siRNA can include a low molecular RNA (for example, tRNA, rRNA, natural RNA molecule such as virus RNA, or synthetic RNA molecule) at the protruding region of one end as long as the siRNA can keep the target gene inhibiting effect. The both ends of the siRNA are not necessarily cleavage structures. That is, the siRNA can have a stem-loop structure wherein one of both ends of the double-strand RNA is linked by a linker RNA. The length of the linker is not limited as long as it does not affect paring in the stem.

In the siRNA of the present invention, the sense RNA strand and/or the antisense RNA strand can contain at least one chemical modification in the sugar region, nucleobase region, or internucleotide structure. Such modification can lead to the prevention of siRNA destruction by nuclease. Any chemical modification that can increase the in vivo stability and biocompatibility of siRNA can be included in this invention. Regarding preferable modifications in the sugar region, such modifications of ribose position 2′ such as 2′-deoxy, 2′-fluoro, 2′-amino, 2′-thio, and 2′-O-alkyl are preferred. Modifications of 2′-O-methyl that can take the place of the normal 2′-OH group in ribonucleotide or modifications in methylene bridge between LNA position 2′ and 4′ are more preferred. The nucleobase can be a modified nucleotide such as 5-bromo-uridine, 5-iodo-uridine, N3-methyl-uridine, 2,6-diaminopurine (DAP), 5-methyl-2′-deoxycytidine, 5-(1-propinyl)-2′-deoxy-uridine (pdU), or 5-(1-propinyl)-2′-deoxycytidine (pdC) or a nucleotide binding to cholesterol. Lastly, one of those preferred modifications in the internucleotide skeleton is achieved by replacing phosphodiester group in the skeleton with phosphorothioate, methylphosphonate, or phosphorodiamidate group, or by constructing the skeleton with N-(2-aminoethyl)-glycine linked by peptide bonds. Such various modifications can be linked to modified morpholino type nucleic acid (nucleotide fixed on morpholoine ring and linked to phosphorodiamidate group) or to PNA (nucleotide fixed on N-(2-aminoethyl)-glycine linked by peptide bonds).

The expression vector to induce the miRNA expression can be selected from the group consisting of pcDNA™ 6.2-GW/EmGFP-mir (Invitrogen), miRNASelect™ pEGP-miR cloning & expression vector or miRNASelect™ pEP-miR cloning & expression vector (CellBiolabs), pmR-ZsGreen1 or pmR-mCherry (Clontech), pCMV-MIR (OriGene), and pLV-miRNA vector (BiOSETTIA), but not limited thereto and any vector capable of inducing the miRNA expression can be used.

The transfection can be performed by using a commercial transfection reagent selected from the group consisting of Lipofectamine, Hilymax (Dojindo), Fugene, jetPEI, Effectene, and DreamFect with the method using calcium-phosphate, cationic polymer, liposome, nano-particle, nucleofection, electroporation, heat shock, or magnetofetion.

The transfected cell line above is characterized by the reduced GSL level.

In the method above, the cell line can be mammalian cells, yeast cells, or insect cells. The mammalian cells are preferably one of those selected from the group consisting of Chinese hamster ovary cells (CHO), HT-1080, human lymphoblastoid, SP2/0 (mouse myeloma), NS0 (mouse myeloma), baby hamster kidney cells (BHK), human embryonic kidney cells (HEK), PERC.6 (human retinal cells), and EC2-1H9, and more preferably Chinese hamster ovary cells (CHO), but not always limited thereto.

To obtain the EC2-1H9 cell line of the present invention capable of mass-producing erythropoietin, the plasmid vector pCMV-dhfr was digested with the restriction enzymes Hind□ and ApaI. The obtained fragment was modified with klenow, to which the EPO genomic gene EpoG was added, resulting in the construction of the expression vector pEpoG-dhfr. The plasmid vector pCMV-dhfr was conjugated with the EPO cDNA gene EpoC, resulting in the construction of the expression vector pEpoC-dhfr. CHO cell line (ATCC CRL 9096) having dhfr mutation was transfected with the expression vector pEpoG-dhfr or pEpoC-dhfr, and as a result EC2, the transformant having human EPO producing activity was prepared. The prepared cells were cultured in |A-MEM supplemented with MTX. The culture medium was prepared by mixing DMEM/F12 (Gibco) containing 10% FBS with 10 μg/ml of hypoxanthine, 10 μg/ml of thymidine, 50 μg/ml of glycine, 587 μg/ml of glutamine, 4.5 mg/ml of glucose, and 100 IU/ml of penicillin-streptomycin (Sigma). The cells were sub-cultured in the prepared culture medium at 37□ in the presence of 5% CO₂. CHO cells having dhfr mutation were collected by using a centrifuge. The cells were inoculated in a 6 cm dish at the density of 5×10⁵ cells/ml. The cells were washed next day twice with OPTI MEM I (Gibco).

5 μg of plasmid was diluted in 1.5 ml of OPTI MEM I and then mixed with 20 μg of Lipofectin diluted as same, which stood 10 minutes before added to CHO cells having dhfr mutation. The cells were cultured in a 37□ 5% CO₂ incubator for 6 hours. 3 ml of DMEM/F12 containing 20% FBS was added thereto, followed by further culture for 24 hours. The culture vessel was treated with 0.25% trypsin, followed by centrifugation. The obtained cells were distributed in a 96-well plate at the density of 2×10⁴ cells/well. IA-MEM (Gibco) supplemented with 550 μg/ml of G418 and dialyzed serum (Gibco 26300-020) 10% was added to the plate, followed by further culture. The medium was replaced every 4 days for 2 week culture. Tens of survived clones were collected. To measure the EPO producing ability of each clone, indirect ELISA using mouse anti-human EPO antibody and peroxidase-conjugated goat anti-mouse IgG antibody (Sigma) was performed (Engvall & Perlman, J. Immunol. 109, 129, 1972). As a result, the cell line EC2 that was confirmed to produce EPO best among those transformants harboring the expression vector pEpoG-dhfr or pEpoC-dhfr was selected.

The present invention provides a recombinant cell line producing a recombinant glycoprotein having a high sialic acid content wherein glycosphingolipid biosynthesis pathway is suppressed.

The present invention also provides a use of the recombinant cell line producing recombinant glycoproteins having a high sialic acid content wherein glycosphingolipid biosynthesis pathway is suppressed.

The glycoprotein herein is preferably selected from the group consisting of erythropoietin, thrombopoietin, alpha-antitrypsin, cholinesterase, chorionic gonadotropin, CTLA4Ig, Factor □, gammaglutamyltransferase, granulocyte colony-stimulating Factor (G-CSF), and luteinizing hormone (LH) which are suitable for the conjugation with sialic acid, and more preferably either erythropoietin or thrombopoietin, but not always limited thereto.

The inhibition of glycosphingolipid biosynthesis pathway is preferably achieved by inhibiting ceramide glucosyltransferase (CGT).

The transfected cell line above is characterized by the reduced GSL level.

The cell line herein can be mammalian cells, yeast cells, or insect cells. The mammalian cells are preferably one of those selected from the group consisting of Chinese hamster ovary cells (CHO), HT-1080, human lymphoblastoid, SP2/0 (mouse myeloma), NS0 (mouse myeloma), baby hamster kidney cells (BHK), human embryonic kidney cells (HEK), PERC.6 (human retinal cells), and EC2-1H9, and more preferably Chinese hamster ovary cells (CHO), but not always limited thereto.

The cell line in this invention displays one of those specific cell morphologies that is suitable for in vitro growth. The cell line can be growing in the established cell culture system and can be proliferated unlimitedly in a provided proper/fresh medium and space. The method for establishing a cell line from the separated cells is well-known to those in the art. In a preferred embodiment of the present invention, the cell line was stable and could be selected by a specific plasmid selection marker containing miRNA inhibiting CGT. Herein, blasticidin was used for the selection.

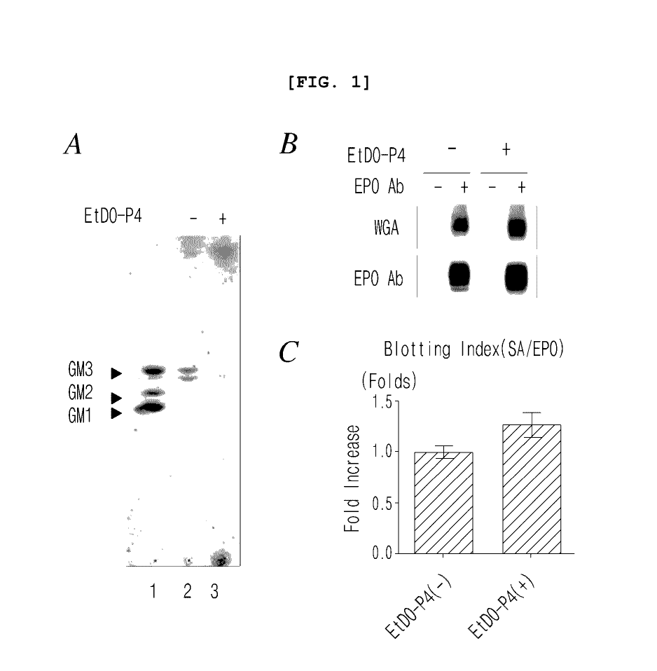

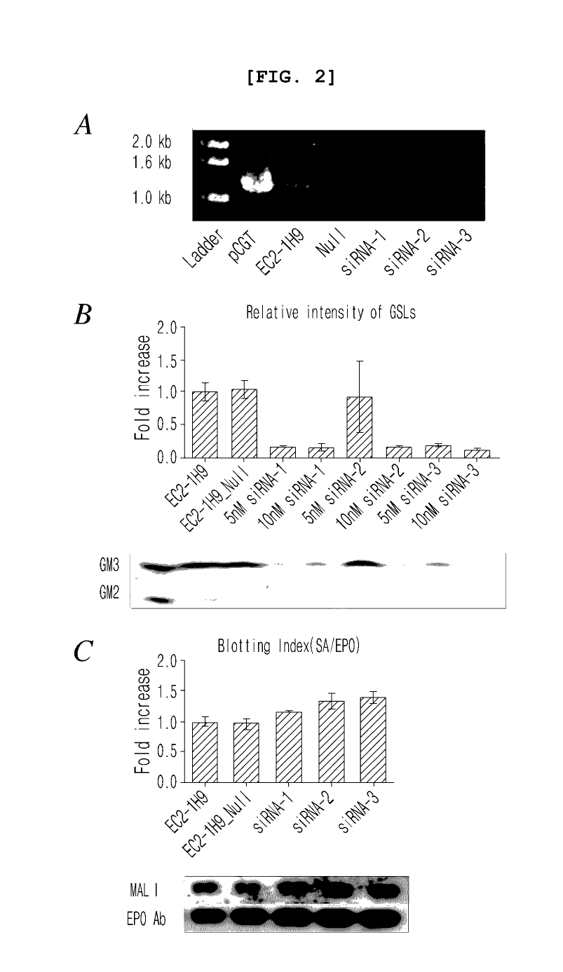

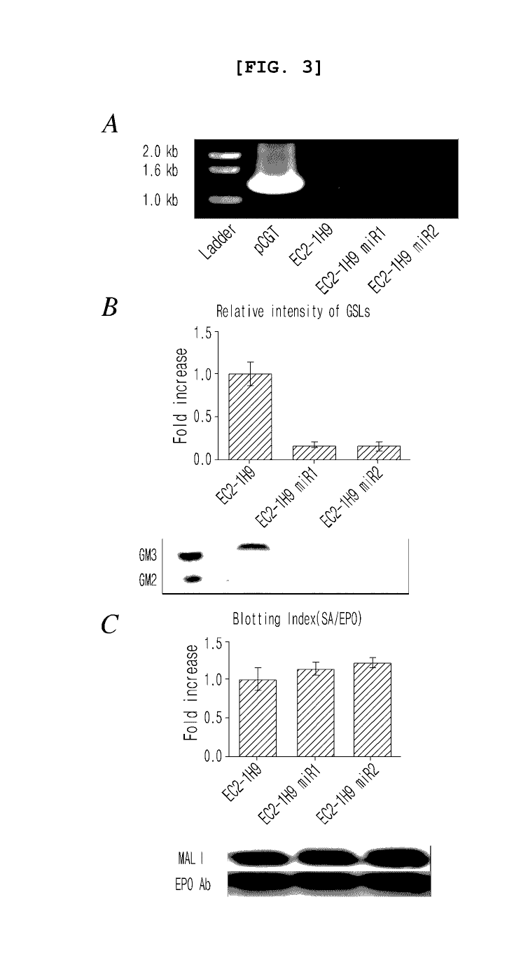

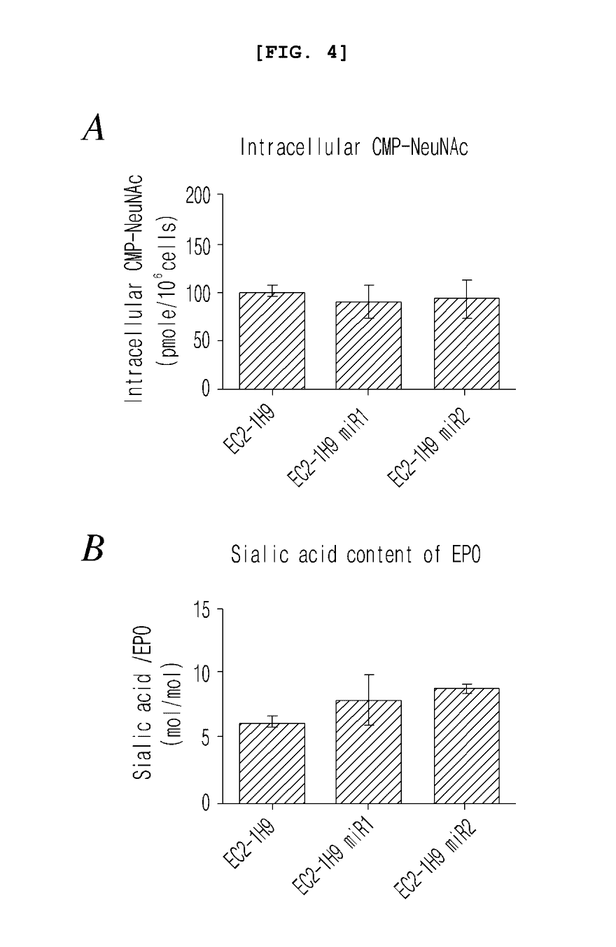

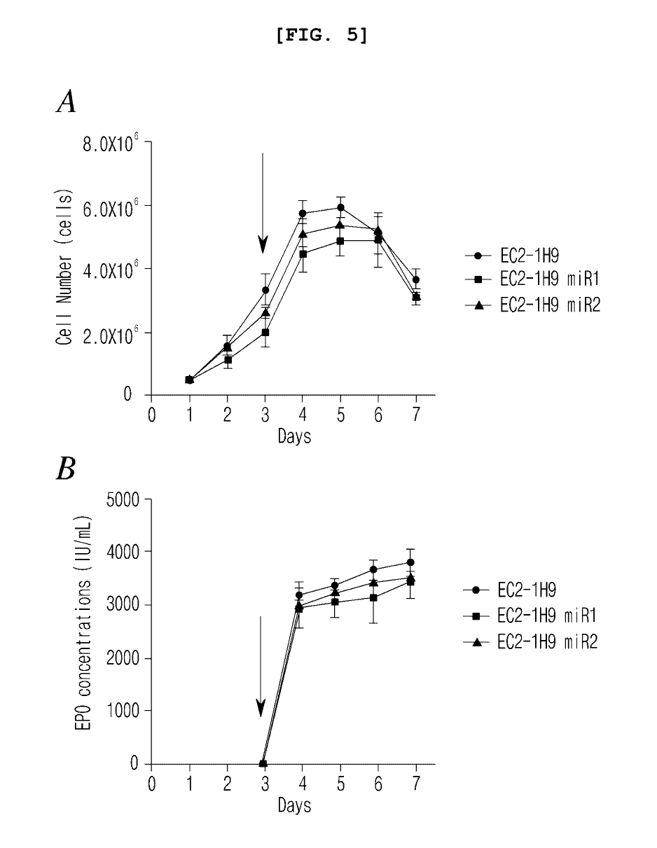

In a preferred embodiment of the present invention, the changes of glycosphingolipid and sialic acid according to the treatment of EtDO-P4 were investigated in the cell line producing recombinant human EPO. As a result, GSL in the recombinant human erythropoietin (rhEpo) was reduced, while sialic acid was increased in the same (see FIG. 1). The changes of glycosphingolipid and sialic acid according to the treatment of CGT-siRNA were also investigated in EC2-1H9 cell line. As a result, the CGT expression was not observed and glycosphingolipid was reduced in the cell line treated with siRNA (see FIG. 2). In the meantime, the sialic acid content was increased in the purified rhEPO. The decrease of GSL was also confirmed in those cell lines wherein CGT was stably inhibited (EC2-1H9-miR 1 and EC2-1H9-miR 2) (see FIG. 3). It was also confirmed that the sialic acid content was increased in the same, compared with that of the control cell line EC2-1H9 (see FIG. 4). The cell growth pattern and the rhEPO production were similar to those of the control cell line (see FIG. 5).

Therefore, the present invention is useful for the prevention and treatment of disease requiring a recombinant therapeutic protein by providing EC2-1H9-miR1 and EC2-1H9-miR2 which characteristically inhibit CGT by using siRNA and miRNA specifically binding to ceramide glucosyltransferase (CGT) and display increased stability of the recombinant therapeutic protein by producing erythropoietin (EPO) having high sialic acid content.

In addition, the present invention provides a method for preparing a recombinant glycoprotein having a high sialic acid content which comprises the following steps:

1) culturing the recombinant cell line; and,

2) separating a glycoprotein having high sialic acid content from the culture solution of step 1).

The glycoprotein herein is preferably selected from the group consisting of erythropoietin, thrombopoietin, alpha-antitrypsin, cholinesterase, chorionic gonadotropin, CTLA4Ig, Factor □, gammaglutamyltransferase, granulocyte colony-stimulating Factor (G-CSF), and luteinizing hormone (LH) which are suitable for the conjugation with sialic acid, and more preferably either erythropoietin or thrombopoietin, but not always limited thereto.

The said erythropoietin of the invention is useful for the treatment or prevention of human CNS or peripheral nervous system disease showing neural or mental symptoms, eye disease, cardiovascular disease, cardiopulmonary disease, respiratory disease, kidney disease, genitourinary disease, gastrointestinal disease, and endocrine & metabolic disease.

The method for separating the glycoprotein above is well-known to those in the art, but immunoaffinity chromatography is preferably used herein.

Therefore, the present invention is useful for the prevention and treatment of such diseases as human CNS or peripheral nervous system disease showing neural or mental symptoms and metabolic disease by providing EC2-1H9-miR1 and EC2-1H9-miR2 which characteristically inhibit CGT by using siRNA and miRNA specifically binding to ceramide glucosyltransferase (CGT) and display increased stability of the recombinant therapeutic protein by producing erythropoietin (EPO) having high sialic acid content.

Practical and presently preferred embodiments of the present invention are illustrative as shown in the following Examples.

However, it will be appreciated that those skilled in the art, on consideration of this disclosure, may make modifications and improvements within the spirit and scope of the present invention.

### Example 1: Cell Line Culture and EtDO-P4 Treatment

EC2-1H9, the CHO cell line producing recombinant human erythropoietin (EPO), was provided from Kangwon National University (Korea). The cells were maintained in MEM-α supplemented with 10% dFBS (dialyzed FBS; SAFC, US), 3.5 g/L glucose, 20 nM MTX (methotrexate, Sigma), and 1% antibiotics-antimycotics solution (Gibco) in the presence of 5% CO₂ at 37□ with humidity. EtDO-P4 (D-threo-1-(3′,4′-ethylenedioxy)-phenyl-2-palmitoylamino-3-pyrrolidino-1-propanol), the glycosphingolipid (GSL) biosynthesis inhibitor, was provided from Dr. James A. Shayman, University of Michigan, USA.

### Example 2: Ceramide Glucosyltransferase Cloning

Chinese hamster (Cricelus griseus) UDP-glucose ceramide glucosyltransferase (UGCG or CGT) was cloned from Chinese hamster ovarian cells (GenBank® accession numbers NM001246692). Total RNA was extracted from Chinese hamster ovarian cells by using TRIzol® reagent (Invitrogen, Carlsbad, Calif.). CGT cDNA strand was synthesized by RT-PCR (AccuPower RT-PCR PreMix; Bioneer). At this time, the forward primer (5′-CTC GAG ATG GCG CTG CT-3′; SEQ. ID. NO: 1) and the reverse primer (5′-TCT AGA TTA TAC ATC TAG GAT TTC CTC TGC-3′; SEQ. ID. NO: 2) were used. The cloned CGT was inserted in the pGEM-T easy vector (PROMEGA, Madison, Wis.), and as a result pCGT was obtained. The gene sequence was identified by di-deoxy sequencing.

### Example 3: Construction of siRNA and miRNA Expression Vectors

The Chinese hamster CGT cDNA sequence of the coding region displayed as high homology as 95.86%, 93.92%, and 92.32% with mouse (GenBank acession no. NM_011673), rat (GenBank accession no. NM_031795), and human (GenBank accession no. NM_003358). The siRNA sequence candidates (siRNA-675, SEQ. ID. NO: 4 and NO: 5; siRNA-926, SEQ. ID. NO: 6 and NO: 7; and siRNA-1098, SEQ. ID. NO: 8 and NO: 9) for the inhibition of Chinese hamster CGT mRNA (GenBank accession no. NM_001246692; SEQ. ID. NO: 3) were synthesized by Bioneer. The oligonucleotides above are shown in Table 1 below. EC2-1H9 cells were transiently transfected with CGT or the negative control siRNA by using a transfection reagent (lipofectamine 2000, Invitrogen) according to the manufacturer's protocol.

For the continuous down-regulation, the oligonucleotide CGT-1098_F (SEQ. ID. NO: 10) and CGT-1098_R (SEQ. ID. NO: 11) listed in Table 1 were used for the expression of miRNA. The produced miR-1098 was inserted in pcDNA™ 6.2-GW/EmGFP-miR vector (Invitrogen), leading to the construction of pcCGT-miR. The vector contained a blasticidin selection gene and the obtained transcript could produce functional miRNA in the target cells.

Experimental Example 1: Investigation of the Effect of EtDO-P4 in the Cell Line Producing Recombinant Human EPO

EC2-1H9, the cell line producing recombinant human EPO (rhEPO), was treated with EtDO-P4, followed by the investigation of the effect of the treatment on glycosphingolipid and sialic acid. To do so, the following experiment was performed.

Particularly, in order to treat EtDO-P4, the cells (5×10⁶) were cultured in 100-mm plates containing MEM-α supplemented with 10% dFBS for 24 hours. The cells were washed twice with serum-free MEM-α. Then, the cells were added with 1 μM EtDO-P4/MEM-α, followed by further culture for 48 hours.

As a result, as shown in FIG. 1, the glycosphingolipid production based on the synthesis of glucosylceramide was inhibited by EtDO-P4, the ceramide glucosyltransferase (CGT) inhibitor (Lee et al. 1999). The treatment of EtDO-P4 reduced completely the major GSLs, GM1, GM2, and GM3, in EC2-1H9 cell line (FIG. 1A). On the contrary, the sialic acid content of recombinant erythropoietin (EPO) was increase by the treatment of EtDO-P4 in EC2-1H9. Wheat germ agglutinin (WGA) lectin blotting confirmed that the content of sialic acid of the purified recombinant human erythropoietin (rhEpo) was increased up to 22% (FIGS. 1B and 1C). The result above indicates that a GSL biosynthesis component such as sialic acid can be useful for the glycoprotein glycosylation for co-substrates (FIG. 1).

Experimental Example 2: Temporary Down-Regulation of CGT by siRNA

To investigate the effect of the treatment of CGT-siRNA on the levels of glycosphingolipid and sialic acid in EC2-1H9 cells, the cells were transfected with siRNA_675 (siRNA1), siRNA_926 (siRNA2), and siRNA_1098 (siRNA3) of Example 3, shown in Table 1 below.

Particularly, EC2-1H9 cells were transfected with three different CGT-siRNAs (siRNA1, siRNA2, and siRNA3) at different concentrations of 5 nM and 10 nM by using Lipofectamine™ LTX and PLUS™ reagents (Invitrogen). The transfected cells were distributed in a 96-well tissue culture plate (Nunc) at the density of 10 cells/well. The cells were cultured in MEM-α (Gibco) supplemented with 10% dFBS (dialyzed FBS; SAFC, US), 3.5 g/L glucose, 20 nM MTX (methotrexate, Sigma), and 1% antibiotics-antimycotics solution (Gibco) in the presence of 5% CO₂ at 37□ with humidity, followed by selecting stable cell lines for 2 weeks. 24 hours after the transfection, total RNA was extracted by using Trisol® reagent. Reverse transcription to cDNA was performed with 1.0 μg of RNA by using AccuPower RT-PCR PreMix (Bioneer). To confirm the mRNA transcript of Chinese hamster CGT by PCR, cDNA was used as a template. To amplify Chinese hamster CGT, the same primers as those used for CGT cloning were used. The amplified gene was separated on 0.8% agarose gel, followed by staining with ethidium bromide for further observation.

Glycosphingolipid (GSL) was sonicated for 30 minutes, followed by extraction using chloroform/methanol (C/M; 2:1, v/v) and isopropanol/hexane/water (IPA/H/W; 55:25:20 v/v/v). The GSL extract was dissolved in 0.1 M NaOH/MeOH, followed by culture for 2 hours at 40□. The extract was neutralized with 1 N HCl, to which hexane was added. The mixture stood for 5 minutes. The lower layer was evaporated in the N2 atmosphere, and the remaining part was dissolved in distilled water. The mixture was applied to SepPak C_{˜}cartridge (Varian, Palo Alto, Calif.). After washed with distilled water, total GSL was extracted with C/M, followed by evaporation. The residue dissolved in C/M was spotted on TLC plate (Silica Gel 60 F-254, Merck), followed by development in C/M/0.2% CaCl₂ (55:40:10, v/v/v). The plate was then stained with 2% orcinol in 2 M H₂SO₄ by spraying.

As a result, as shown in FIG. 2, the pCGT vector constructed in Example 2 was used as the positive control (FIG. 2A, lane 1) and the Chinese hamster CGT transcript was detected in EC2-1H9 control cells (FIG. 2A, lane 2). The expression of CGT was not observed in the siRNA treated cells. Glycosphingolipid (GSL) was reduced in the siRNA treated cells (FIG. 2B). It was also confirmed from the investigation of comparative strength on Maackia Amurensis Lectin I (MAL I) blot that the content of sialic acid in the purified rhEPO was increased up to 22% (FIG. 2C). To construct the miRNA expression vector based on RT-PCR and lectin blotting, a siRNA candidate group was selected.

TABLE 1

| | Oligo- | | |
|---|---|---|---|
| | nucleotide | Oligonucleotide Sequence | cDNA |
| Name | Name | (target sequence) | site |
| siRNA- | S-siRNA | 5'-UGAUAGCCUUUGCUCAGUACA- | 677 |
| 675 | (SEQ. ID. | 3' | |
| | NO: 4) | | |
| | A-siRNA | 5'-UGUACUGAGCAAAGGCUAUCA- | |
| | (SEQ. ID. | 3' | |
| | NO: 5) | | |
| siRNA- | S-siRNA | 5'-GUGUUCAGAUGGGAUAUCAUG- | 928 |
| 926 | (SEQ. ID. | 3' | |
| | NO: 6) | | |
| | A-siRNA | 5'-CAUGAUAUCCCAUCUGAACAC- | |
| | (SEQ. ID. | 3' | |
| | NO: 7) | | |
| siRNA- | S-siRNA | 5'-CAUUAUGGGACCCUACUAUAA- | 1100 |
| 1098 | (SEQ. ID. | 3' | |
| | NO: 8) | | |
| | A-siRNA | 5'-UUAUAGUAGGGUCCCAUAAUG- | |
| | (SEQ. ID. | 3' | |
| | NO: 9) | | |
| miRNA- | CGT-1098F | 5'-TGCTGTTATAGTAGGGTCCCATAAT | |
| 1098 | (SEQ. ID. | GGTTTTGGCCACTGACTGACCATTA | |
| | NO: 10) | TGGCCCTACTATAA-3' | |
| | CGT-1098R | 5'-CCTGTTATAGTAGGGCCATAATGGT | |
| | (SEQ. ID. | CAGTCAGTGGCCAAAACCATTATGG | |
| | NO: 11) | GACCCTACTATAAC-3' | |

### Experimental Example 3: Construction of Stable Cell Line Wherein CGT is Continually Down-Regulated

To investigate the changes of glycosphingolipid and sialylation in the stable cell line displaying the continuous down-regulation of CGT by analyzing glycan, the following experiment was performed by the same manner as described in Experimental Example 2.

Particularly, EC2-1H9 cells were transfected with pcCGT-miRNA constructed in Example 3 by using Lipofectamine™ LTX and PLUS™ (Invitrogen). The stable cell line over-expressing CGT miRNA was selected by using blasticidin. To detect the expression of the transformed gene, total RNA was extracted from each transcript. RT-PCR was performed to detect Chinese hamster CGT transcript.

As a result, as shown in FIG. 3, the Chinese hamster CGT transcript was detected in EC2-1H9 control cells at a low concentration (FIG. 3A, lane 3). At this time, pcCGT-miR vector was used as the positive control (FIG. 3A, lane 1). Two clones (EC2-1H9-miR 1 and EC2-1H9-miR 2) did not show CGT gene expression (FIG. 3A, lane 4 and lane 5). Glycosphingolipid was reduced in the siRNA treated cells (EC2-1H9-miR 1 and EC2-1H9-miR 2) (FIG. 3B). Sialic acid was increased in the siRNA treated cells (FIG. 3C).

### Experimental Example 4: Quantitative Analysis of CMP-Sialic Acid in the Stable Cell Line

To investigate the expression of sialuria-mutated rat GNE/MNK in the stable cell line, intracellular CMP-sialic acid was quantitatively analyzed as follows.

Particularly, 1.0×10⁷ cells were dissolved in ice-cold 75% (v/v) ethanol by using a sonic cell disruptor (Vibra cell 130; Sonics & Materials). The soluble part was centrifuged at 13,000 rpm for 10 minutes at 4□, followed by freeze-drying. To stabilize CMP-sialic acid, the prepared sample was resuspended in 120 μl of 40 mM phosphate buffer at pH 9.2, followed by centrifugation. The supernatant was filtered with a membrane (Microcon®; Millipore) through which only up to 10,000 MW could pass. Sugar nucleotide was separated on CarboPac PA-1 column (Dionex). OD was measured with an absorbance detector at Abs₂₆₀ (model 486 tunable UV visible absorbance detector). The level of intracellular CMP-sialic acid was standardized to cell number.

As a result as shown in FIG. 4A, there was no difference of the intracellular CMP-NeuNAc content between the control cell line and the transfected cell line (FIG. 4A).

### Experimental Example 5: Analysis of the Sialic Acid Content of rhEPO in the Stable Cell Line

To measure the sialic acid content of the purified rhEPO in the stable cell line, the following experiment was performed using OPD-labeling method.

Particularly, EC2-1H9 cells and the selected cell line were distributed in T-175 cm² culture flask (Nunc) containing MEM-α supplemented with 10% (v/v) dFBS, 3.5 g/L glucose, 1% (v/v) Ab-Am solution, and 20 nM MTX, at the density of 5.0×10⁶ cells. Three days later, the culture medium was replaced with a serum-free medium (CHO-S-SFM II; Gibco), followed by further culture for 2 days. The supernatant containing the recombinant human EPO (rhEPO) was collected and filtered through 0.45 μm membrane. To separate rhEPO, the filtered culture medium was concentrated and the medium was replaced with PBS by using ultrafiltration (AmiconUltra; Millipore). The rhEPO was purified by immune-affinity chromatography composed of mouse anti-human EPO monoclonal antibody (R&D systems) conjugated CNBr-activated Sepharose 4B (Amersham Biosciences). The sample was loaded and washed with PBS. The conjugated rhEPO was eluted by using 0.1 M glycine/0.5 M NaCl, pH 2.8. The eluent was neutralized right away with 1.0 M Tris/HCl, pH 9.0. The purified rhEPO was concentrated, followed by dialysis with distilled water by using ultrafiltration (AmiconUltra; Millipore). The concentration of rhEPO was determined by using Quant-iT™ protein analysis kit (Invitrogen), followed by freeze-drying.

For the quantitative analysis of sialic acid in rhEPO, the sialic acid content in rhEPO was determined by using OPD-labeling method (Anumula 1995). Particularly, sialic acid was separated from the purified rhEPO in 0.5 M NaHSO₄ at 80□ for 20 minutes, and then induced with OPD (o-phenylenediamine-2HCl; Sigma) at 80□ for 40 minutes. The OPD-labeled sialic acid was separated on C₁₈ reverse Cis column (Shim-pack CLC-ODS; Shimadzu) by using HPLC, and then detected by using a fluorescence detector (model 474; Waters) (emission wavelength: 230 nm, excitation wavelength: 425 nm). Human EPO has an O-linked glycosylated region (two sialic acid residues) and three N-linked glycosylated regions (4 sialic acid residues), so that up to 14 mol of sialic acid can be linked to 1 mol of rhEPO.

As a result, as shown in FIG. 4B, the sialic acid consents of rhEPO in EC2-1H9-miR1 and miR2 cell lines were respectively 7.8 and 8.7 sialic acid mol/rhEPO mol. The result indicates, compared with the sialic acid content in the EC2-1H9 control cell line, that the sialic acid content was respectively increased approximately 26.5% and 4.01% therein (FIG. 4B).

### Experimental Example 6: Confirmation of Cell Growth and rhEPO Production

To evaluate the effect of gene over-expression on cell growth pattern and rhEPO production, cell growth and rhEPO production were first investigated.

Particularly, the cells growing exponentially were distributed in a 6-well culture plate (Nunc) at the density of 0.5×10⁶ cells/well. 3 days after the culture, the culture medium was replaced with a serum-free medium (CHO-S-SFM II; Gibco), followed by the confirmation of rhEPO production (FIGS. 5A and 5B, arrows). Cells were separated by using trypsin and then stained with trypan blue. The live cells were counted with a hemocytometer. The discharged rhEPO concentration was measured by ELISA. The 96-well immunoplate (Nunc) was coated with rabbit anti-human EPO polyclonal antibody (Sigma) at 4□ for 18 hours, followed by blocking with 2% BSA at room temperature for 1 hour in TBS-T (10 mM Tris-HCl containing 150 mM NaCl and 0.05% Tween 20, pH 7.4). After washing the plate with TBS-T three times, the serially diluted rhEPO standard and the cultured medium supernatant were loaded thereto for 2 hours at 37□, followed by culture. After washing the plate with TBS-T three times, the plate was co-cultured with HRP-conjugated goat anti-mouse IgG secondary antibody (1:2,000) at 37□ for 30 minutes, followed by washing with TBS-T three times again. 100 μl of TMB liquid substrate system for ELISA (3,3′, 5,5′tetramethylbenzidine; Sigma) was added to each well, and then OD₃₇₀ was measured with a microplate reader (Infinite M200 Megellan, Tecan).

The results are shown in FIG. 5 (FIG. 5).

Those skilled in the art will appreciate that the conceptions and specific embodiments disclosed in the foregoing description may be readily utilized as a basis for modifying or designing other embodiments for carrying out the same purposes of the present invention. Those skilled in the art will also appreciate that such equivalent embodiments do not depart from the spirit and scope of the invention as set forth in the appended Claims.

1. A method for preparing a cell line producing a glycoprotein with increased sialic acid content, which comprises the step of inhibiting glycosphingolipid (GSL) biosynthesis.

2. The method for preparing a cell line producing a glycoprotein with increased sialic acid content according to claim 1, wherein the glycoprotein is selected from the group consisting of erythropoietin, thrombopoietin, alpha-antitrypsin, cholinesterase, chorionic gonadotropin, cytotoxic T-lymphocyte-associated protein 4 Ig (CTLA4Ig), Factor VIII, gammaglutamyltransferase, granulocyte colony-stimulating Factor (G-CSF), and luteinizing hormone (LH).

3. The method for preparing a cell line producing a glycoprotein with increased sialic acid content according to claim 1, wherein the inhibition of glycosphingolipid biosynthesis pathway is achieved by suppressing activity of ceramide glucosyltransferase (CGT).

4. The method for preparing a cell line producing a glycoprotein with increased sialic acid content according to claim 3, wherein the ceramide glucosyltransferase is composed of the nucleotide sequence represented by SEQ. ID. NO: 3.

5. The method for preparing a cell line producing a glycoprotein with increased sialic acid content according to claim 3, wherein the suppression of activity of ceramide glucosyltransferase is achieved through the treatment of a ceramide glucosyltransferase inhibitor or through the transfection with any sequence selected from the group consisting of antisense nucleotide, siRNA, shRNA, and miRNA binding to ceramide glucosyltransferase mRNA.

6. The method for preparing a cell line producing a glycoprotein with increased sialic acid content according to claim 5, wherein the siRNA is composed of one of those sequences selected from the group consisting of the sequences represented by SEQ. ID. NO: 4˜NO: 9.

7. The method for preparing a cell line producing a glycoprotein with increased sialic acid content according to claim 5, wherein the miRNA is composed of the nucleotide sequence either represented by SEQ. ID. NO: 10 or NO: 11.

8. The method for preparing a cell line producing a glycoprotein with increased sialic acid content according to claim 5, wherein the cell line is characterized by the reduced GSL level.

9. The method for preparing a cell line producing a glycoprotein with increased sialic acid content according to claim 1, wherein the cell line is selected from the group consisting of mammalian cells, yeast cells, and insect cells.

10. The method for preparing a cell line producing a glycoprotein with increased sialic acid content according to claim 9, wherein the cell is selected from the group consisting of Chinese hamster ovary cells (CHO), HT-1080, human lymphoblastoid, SP2/0 (mouse myeloma), NS0 (mouse myeloma), baby hamster kidney cells (BHK), human embryonic kidney cells (HEK), and PERC.6 (human retinal cells).

11. A cell line producing a glycoprotein with increased sialic acid content wherein the glycosphingolipid biosynthesis pathway is suppressed.

12. The cell line producing a glycoprotein with increased sialic acid content according to claim 11, wherein the glycoprotein is selected from the group consisting of erythropoietin, thrombopoietin, alpha-antitrypsin, cholinesterase, chorionic gonadotropin, cytotoxic T-lymphocyte-associated protein 4 Ig(CTLA4Ig), Factor VIII, gammaglutamyltransferase, granulocyte colony-stimulating Factor (G-CSF), and luteinizing hormone (LH).

13. The cell line producing a glycoprotein with increased sialic acid content according to claim 11, wherein the inhibition of glycosphingolipid biosynthesis pathway is achieved by suppressing activity of ceramide glucosyltransferase (CGT).

14. A method for preparing a glycoprotein with increased sialic acid content which comprises the following steps:
1) culturing the cell line of claim 11; and,
2) separating a glycoprotein with increased sialic acid content from the culture solution of step 1).

15. A method for preparing a glycoprotein with increased sialic acid content according to claim 14, wherein the glycoprotein is selected from the group consisting of erythropoietin, thrombopoietin, alpha-antitrypsin, cholinesterase, chorionic gonadotropin, cytotoxic T-lymphocyte-associated protein 4 Ig(CTLA4Ig), Factor VIII, gammaglutamyltransferase, granulocyte colony-stimulating Factor (G-CSF), and luteinizing hormone (LH).

16. (canceled)
